(19) United States
(12) Patent Application Publication
(10) Pub. No.: US 2023/0330005 A1
(43) Pub. Date: Oct. 19, 2023

(54) SELF-ASSEMBLED COMPLEX CONTAINING CALCIUM ION

(71) Applicant: Korea University Research and Business Foundation, Seoul (KR)

(72) Inventors: Heemin KANG, Seoul (KR); Sung-Gue LEE, Siheung-si (KR); Na-Yeon KANG, Seoul (KR); Yu-Ri KIM, Seoul (KR); Gun-Hyu BAE, Seoul (KR); Da-Hee KIM, Seoul (KR); Seong-Yeol KIM, Seoul (KR)

(73) Assignee: Korea University Research and Business Foundation, Seoul (KR)

(21) Appl. No.: 17/989,930

(22) Filed: Nov. 18, 2022

(30) Foreign Application Priority Data
Apr. 13, 2022 (KR) 10-2022-0046022
Aug. 5, 2022 (KR) 10-2022-0097805

(51) Int. Cl. A61K 9/00; A61K 45/06

(52) U.S. Cl. CPC A61K 9/0002; A61K 45/06

(57) ABSTRACT

Disclosed is a self-assembled complex containing a calcium ion that includes a calcium ion; and at least one ligand, where the calcium ion and the ligand participate in a reversibly self-assembly or self-disassembly. The shape of the self-assembled complex varies depending on the type of the ligand, or the mixing ratio of the ligand when there are a plurality of ligands.

### CROSS-REFERENCE TO RELATED APPLICATIONS

This application claims priority to and the benefit of Korean Patent Application No. 10-2022-0046022 filed on Apr. 13, 2022 and Korean Patent Application No. 10-2022-0097805 filed on Aug. 5, 2022, in the Korean Intellectual Property Office, the entire contents of which are incorporated herein by reference.

### BACKGROUND OF THE INVENTION

### (A) Field of the Invention

The present invention relates to a self-assembled complex containing a calcium ion, and particularly to a self-assembled complex containing a calcium ion and a ligand to participate in self-assembly and self-disassembly.

### (B) Description of the Related Art

Substances, such as hydroxyapatite (HA), β-tri-calcium phosphate (TCP) and biphasic HA-TCP, containing calcium and phosphate and being available as a bone regeneration filler require external forces like microwave or heat during their manufacturing process, incurring additional production costs associated with the facility and process necessary for that reason.

The above-mentioned bone regeneration filler requires chemical additives in addition to essential ingredients to form a shape, which may cause problems in biocompatibility when in use for a living body and involve unnecessary efforts in the manufacture and the removal of unnecessary additives.

Calcium ions are essential for bone regeneration, and humans consume most of the necessary calcium through foods. When it comes to the bone regeneration treatment required due to a bone fracture, an external calcium supply is not needed for patients with minor injuries, but essential for the elderly or severely injured patients.

Adenosine monophosphate is effective for bone regeneration by promoting the differentiation and mineralization of osteoblasts in vivo.

Phosphates can form a complex through a coordination bond with a metal, including calcium.

Substances, such as hydroxyapatite (HA), β-tri-calcium phosphate (TCP) and biphasic HA-TCP, available for the bone regeneration treatment have different bone regeneration capabilities depending on their shape, porosity and surface roughness.

Korean Patent Application Laid-Open No. 10-2018-0009012 discloses a technology related to a self-assembled nanostructure applicable to a living body, but does not mention the differentiation of functions according to characteristics such as the shape of the material.

### SUMMARY OF THE INVENTION

### Technical Problem

It is an object of the present invention to form a self-assembled structure using a substance found in a living body and control the self-disassembly rate of the structure by adjusting the components of the structure, thereby allowing a control of the delivery rate of an active ingredient and a morphological change of the structure.

### Technical Solution

In accordance with one aspect of the present invention, the embodiments of the present invention may include a self-assembled complex containing a calcium ion that includes a calcium ion; and at least one ligand, where the calcium ion and the ligand participate in a reversibly self-assembly or self-disassembly. The shape of the self-assembled complex varies depending on the type of the ligand, or the mixing ratio of the ligand when there are a plurality of ligands.

In an embodiment, the shape of the self-assembled complex may change to a needle, fibrils, and a sphere in sequence with an increase in the concentration of the ligand.

In an embodiment, the needle and the fibrils may have a major axis and a minor axis. Here, the needle has a prolonged shape in which the major axis is at least 40- to 60-fold longer than the minor axis. The minor axis of the fibrils is shorter than that of the needle. The fibrils have a plurality of branch portions formed on the surface thereof. And, the sphere has an average diameter of 0.1 to 10 μm.

In an embodiment, the average diameter of the sphere may be less than the length of the major axis of the needle or the fibrils. The average diameter of the sphere may be greater than the length of the minor axis of the needle or the fibrils.

In an embodiment, the ligand may include at least any one of phosphate and phosphonate.

In an embodiment, the ligand may include at least any one selected from the group consisting of AMP, ADP, ATP, TMP, TDP, TTP, CMP, CDP, CTP, GMP, GDP, GTP, UMP, UDP, UTP, DNA, RNA, AEP (2-aminoethylphosphonic acid), TNA (threose nucleic acid), GNA (glycol nucleic acid), HNA (1,5-anhydrohexitol nucleic acid), ANA (1,5-anhydroatritol nucleic acid), FANA (2′-deoxy-2′-fluoroarabino nucleic acid), and CeNa (cyclohexenyl nucleic acid).

In an embodiment, the ligand may be AMP, and the self-assembled complex may be formed in the shape of a needle.

In an embodiment, the ligand may be a combination of AMP and ATP, and the AMP and the ATP may be included at a molar concentration ratio of 99.99:0.01 to 99.7:0.3. The self-assembled complex may be formed in the shape of fibrils.

In an embodiment, the ligand may be a combination of AMP and ATP, and the AMP and the ATP may be included at a molar concentration ratio of 99.5:0.5 to 96:4. The self-assembled complex may be formed in the shape of a sphere.

In an embodiment, the self-assembled complex may be formed by at least any one of π-π interaction and hydrogen bonding between the adjacent ligands; or coordination bonding between the calcium ion and the ligand.

In an embodiment, the calcium ion and the ligand may be included at a molar concentration ratio of 5:1 to 1:1.

In an embodiment, the self-disassembly may be accelerated under conditions with a buffer solution.

In an embodiment, the self-assembled complex may be self-disassembled for 3 to 40 days in vivo or in vitro.

In an embodiment, the self-assembled complex may further include an active ingredient. The active ingredient may be supported in the self-assembled complex.

In an embodiment, the active ingredient may be at least any one selected from the group consisting of adenosine, guanosine, uridine, cytidine, doxorubicin, a drug, a protein, an organic acid, an organic base, a fragrance, and a dye.

In an embodiment, the active ingredient may be released through a self-disassembly of the self-assembled complex. The active ingredient may be released continuously over a release time.

In an embodiment, the release time may be 3 to 40 days.

In an embodiment, the self-assembled complex may be self-disassembled to release at least any one of the calcium ion and the ligand over a release time. The release time may be adjusted by the mixing ratio of the ligand.

In an embodiment, the self-assembled complex may be formed in the shape of a needle. The major axis of the needle is perpendicular to a fiber sheet to enable a delivery of the calcium ion.

In an embodiment, the self-assembled complex may be used as at least any one selected from a bond regeneration material, an artificial bone material, an in-vivo substance delivery carrier, and an in-vitro substance delivery carrier.

### Effects of Invention

The present invention as described above provides a self-assembled complex containing a calcium ion.

According to the present invention, it is also possible to form a self-assembled structure using a substance found in a living body and control the self-disassembly rate of the structure by adjusting the components of the structure, thereby allowing a control of the delivery rate of an active ingredient and a morphological change of the structure.

### BRIEF DESCRIPTION OF DRAWINGS

FIG. 1 is a schematic diagram showing the self-assembly process of a calcium ion and a ligand according to an embodiment of the present invention.

FIGS. 2 and 3 present SEM images of the self-assembled complexes prepared with the different mixing ratios of AMP and ATP used as ligands according to an embodiment of the present invention.

FIGS. 4, 5 and 6 present the experimental results for a substance delivery of the self-assembled complexes prepared with the different mixing ratios of AMP and ATP used as ligands according to an embodiment of the present invention.

### DETAILED DESCRIPTION OF THE EMBODIMENTS

### Drawings

The details of other embodiments are included in the detailed description and drawings.

The advantages and features of the present invention and methods to achieve these will be more clearly understood from the following detailed description taken in conjunction with the accompanying drawings. However, the present invention is not limited to the embodiments disclosed below, but may be implemented in a variety of different forms. Unless otherwise specified in the following description, all numbers, values and/or expressions indicating the components, reaction conditions, and contents of components in the present invention are modified in all instances by the term “about”, as these numbers are inherently approximations reflecting the various uncertainties of measurements arising out of obtaining such values, among others. Also, in disclosing numerical ranges in this description, such ranges are continuous and inclusive of all values from the minimum to the maximum, unless otherwise specified. Moreover, when such ranges refer to integers, all integers ranging from the minimum to the maximum are included, unless otherwise specified.

When a range is defined for a variable in the present invention, the variable will be understood to include all values within the defined range, including the recited endpoints of the range. For example, a range of “5 to 10” includes the values of 5, 6, 7, 8, 9, and 10, as well as any sub-ranges, such as 6 to 10, 7 to 10, 6 to 9, and 7 to 9. It will be also construed as including any value between integers that are appropriate for the scope of the defined range, such as 5.5, 6.5, 7.5, 5.5 to 8.5, and 6.5 to 9. For example, a range of “10 to 30%” includes values of 10%, 11%, 12%, 13%, etc. and all integers up to 30%, as well as any sub-range, such as 10 to 15%, 12 to 18%, and 20 to 30%, and any value between reasonable integers within the scope of the defined range, such as 10.5%, 15.5%, and 25.5%.

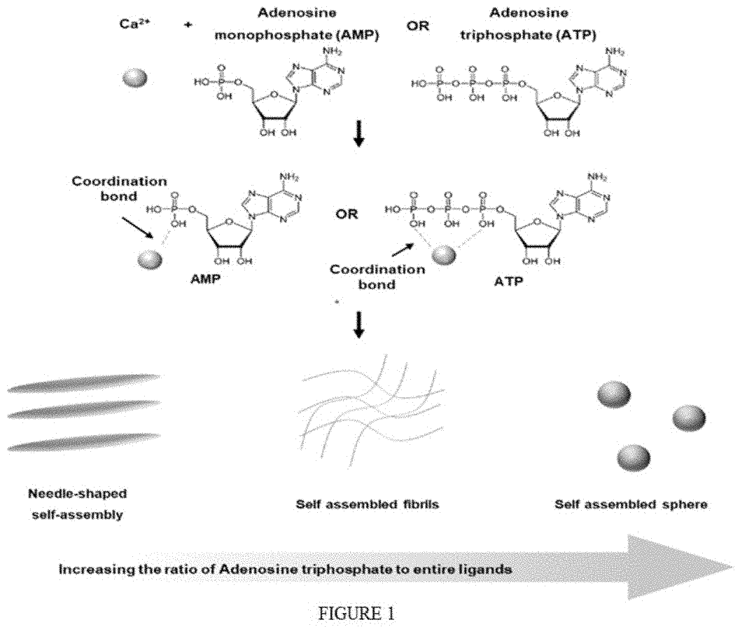

FIG. 1 is a schematic diagram showing the self-assembly process of a self-assembled complex containing a calcium ion according to an embodiment of the present invention.

For example, the calcium ion (Ca²⁺) can form a coordination bond with the phosphate moiety of AMP or ATP used as a ligand to produce a metal complex. If not shown in the figure, the base moiety of the ligand contains an aromatic ring, so π-π interactions can be formed between the aromatic rings. If not shown in the figure, a water molecule can be bound to the calcium ion and to the nitrogen of the base moiety to form hydrogen bonds.

In that manner, the self-assembled complex may be formed to include coordination bonding and π-π interaction between the calcium ion and the ligand. And at least one of the coordination bonding and π-π interaction may be used to form the self-assembled complex.

In one aspect of the present invention, the embodiments of the present invention may include a self-assembled complex containing a calcium ion that includes a calcium ion; and at least one ligand, where the calcium ion and the ligand are reversibly self-assembled or self-disassembled. The shape of the self-assembled complex varies depending on the type of the ligand, or the mixing ratio of the ligand when there are a plurality of ligands.

The shape of the self-assembled complex may change to a needle, fibrils, and a sphere in sequence depending on the mixing ratio of the ligand. For example, a needle and fibrils, or fibrils and a sphere may coexist according to the mixing ratio of the ligand.

The needle and the fibrils have a major axis and a minor axis. Here, the needle has a prolonged shape in which the major axis is at least 40- to 60-fold longer than the minor axis. The minor axis of the fibrils is shorter than that of the needle. The fibrils have a plurality of branch portions formed on the surface thereof. And, the sphere has an average diameter of 0.1 to 10 μm.

In the case that the self-assembled complex is changed in shape from a needle to fibrils, for example, the minor axis of the fibrils may be shorter than that of the needle. Besides, the needle is provided to have an approximately smooth outer surface in the direction of the major axis, and the fibrils have branch portions in the form of scale hairs in the direction of the major axis.

The self-assembled complex may also be changed in shape from the fibrils to a sphere by varying the mixing ratio of the ligand. The average diameter of the sphere may be greater than the length of the minor axis of the needle and less than the length of the major axis of the needle.

The average diameter of the sphere may be less than the length of the major axis of the needle or the fibrils and greater than the length of the minor axis of the needle or the fibrils.

The fibrils may be formed by releasing the condensation of the self-assembled complex in the form of the needle according to the mixing ratio of the ligand. The fibrils may have a longer major axis and a shorter minor axis than the needle.

The surface of the fibrils may be shaped like a branch to form a branch portion.

Depending on the mixing ratio of the ligand, the self-assembled complex may be changed in shape from fibrils to a sphere. As the negative charge of the ligand increases, for example, the fibrils become unstable to have their shape disrupted, and spherical particles can be formed.

Depending on the mixing ratio of the ligand, the needle and the fibrils may exist separately or simultaneously. Also, depending on the mixing ratio of the ligand, the fibrils and the sphere may exist separately or simultaneously.

The ligand may be a substance capable of forming a coordination bond with the calcium ion and participating in the self-assembly. The ligand may form a bonding with the calcium ion to make a metal complex.

The ligand may include at least any one of phosphate and phosphonate. The phosphate or phosphonate may be a moiety capable of forming a coordination bond with the calcium ion.

For example, the ligand may be at least any one selected from the group consisting of adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), DNA, RNA, 2-aminoethylphosphonic acid (AEP), threose nucleic acid (TNA), glycol nucleic acid (GNA), 1,5-anhydrohexitol nucleic acid (HNA), 1,5-anhydroatritol nucleic acid (ANA), 2′-deoxy-2′-fluoroarabino nucleic acid (FANA), and cyclohexenyl nucleic acid (CeNA).

The calcium ion may form a coordination bond with an electron-rich moiety, such as the phosphate moiety of the ligand, to make a metal complex.

For example, the ligand may be AMP and the self-assembled complex may be formed in the shape of a needle. If including AMP alone as the ligand, the self-assembled complex may be shaped like a needle.

For example, the ligand may be a combination of AMP and ATP, where the AMP and the ATP are included at a molar concentration ratio of 99.99:0.01 to 99.7:0.3, and the self-assembled complex may be formed in the shape of fibrils. Preferably, the ligand may include the AMP and the ATP at a molar concentration ratio of 99.99:0.01 to 99.5:0.5, and the self-assembled complex may be shaped like fibrils.

For example, the ligand may be a combination of AMP and ATP, where the AMP and the ATP are included at a molar concentration ratio of 99.5:0.5 to 0.01:99.99, and the self-assembled complex may be formed in the shape of a sphere. The ligand may include the AMP and the ATP at a molar concentration ratio of 99.5:0.5 to 96:4, and the self-assembled complex may be spherically formed. Preferably, the ligand may include the AMP and the ATP at a molar concentration ratio of 99.5:0.5 to 95:5, and the self-assembled complex may be spherically formed.

The self-assembled complex may be formed by at least any one of π-π interaction and hydrogen bonding between the adjacent ligands; or a coordination bond between the calcium ion and the ligand.

The self-assembled complex may be formed by at least any one of a coordination bond, π-π interaction and hydrogen bonding between the calcium ion and the ligand that are adjacent to each other. In the self-assembled complex, the calcium ion and the ligand can be densely adhered to each other by the interactions or bonding. Hence, the self-assembled complex may be formed by dense aggregation of the calcium ion and the ligand.

Depending on the type and mixing ratio of the ligand, the self-assembled complex may be formed more densely or loosely. The mixing ratio may be a molar concentration ratio.

For example, with an increase in the mixing ratio of ATP in the ligand, the negative charge of the self-assembled complex increases, causing the self-assembled complex to form more densely.

The calcium ion and the ligand may be included at a molar concentration ratio of 5:1 to 1:1.

The self-assembled complex may be self-assembled and self-disassembled in a spontaneous manner. The self-disassembly may be accelerated under conditions with a buffer solution. The buffer solution may be designed to create an environment similar to the in-vivo environment. The buffer solution may exchange ions with the components of the self-assembled complex to promote the disassembly of the self-assembled complex.

In the presence of the buffer solution, the self-assembled complex may be self-disassembled over 3 to 40 days.

The self-assembled complex may further include an active ingredient, and the active ingredient may be supported in the self-assembled complex. The active ingredient may become supported inside the self-assembled complex as being incorporated into the self-assembled complex while the self-assembled complex is being self-assembled.

The self-assembled complex may release the active ingredient when it is self-disassembled. It is therefore possible to control the release time and rate of the active ingredient by adjusting the self-disassembly time of the self-assembled complex. The self-disassembly rate of the self-assembled complex may vary by the type of the ligand. The self-assembled complex may be self-disassembled at a defined rate sequentially from the surface thereof, which allows the active ingredient to be continuously released at a constant rate over the self-disassembly time of the self-assembled complex. In other words, the active ingredient may be released constantly, i.e., in a sustained manner over a period of time.

Examples of the active ingredient may include, but are not limited to, at least any one selected from adenosine, guanosine, uridine, cytidine, doxorubicin, a drug, a protein, an organic acid, an organic base, a fragrance, and a dye.

When the self-assembled complex is self-disassembled, it may release at least any one of the calcium ion and the ligand, as well as the active ingredient over a release time. The release time may be adjusted by the mixing ratio of the ligand. Accordingly, the self-assembled complex can be used for delivery of the calcium ion or the ligand into a living body.

For example, the release time may range from 3 days to 40 days.

When formed in the shape of a needle, the self-assembled complex according to this embodiment of the present invention may be used as a patch or the like. Specifically, the needle-shaped self-assembled complex can be fixed to a sheet form, such as a polymer or unwoven fabric sheet, which may be used as a microneedle patch or the like.

If being in the shape of a needle, the self-assembled complex may be formed to have such a rigidity as to penetrate the epidermal layer of the skin and used as a needle for a microneedle patch. In the case of being used as a microneedle, the self-assembled complex may be applicable to cosmetic or medical devices to enhance the delivery efficiency of a substance/drug.

The self-assembled complex may be used as at least any one selected from a bond regeneration material, an artificial bone material, an in-vivo substance delivery carrier, and an in-vitro substance delivery carrier.

Hereinafter, a description will be given as to the examples and comparative examples of the present invention. The following examples are given only to illustrate preferred embodiments of the present invention and are not to be construed to limit the scope of the present invention.

### PREPARATION EXAMPLES

1. Preparation of Self-Assembled Complex

Calcium chloride, adenosine monophosphate (AMP) and adenosine triphosphate (ATP) were separately dissolved in ultrapure water to give a final concentration of 100 mM. The stock solution was put in a conical tube, and a calculated amount of ultrapure water was added. To the stock solution were added the aqueous solutions of adenosine phosphates and the aqueous solution of calcium ion in order. The mixed solutions were blended well by vortexing and stood for 36 hours to bring about a reaction.

2. Supporting Active Ingredient in Self-Assembled Complex

The self-assembled complex was synthesized so that a drug such as adenosine was supported therein. First, a weighed amount of adenosine was put in a conical tube or a glass bottle, followed by adding ultrapure water and finally AMP. After mixing well, a solution containing calcium ion was added, and the resultant mixed solution was stood for 36 hours. After the completion of the reaction, in order to remove unreacted substances and obtain a self-assembled complex, a centrifugation was performed at 10,000 rpm for 10 minutes to make the self-assembled complex settle down, and the supernatant was discarded. Ultrapure water having a volume equal to the original volume was added, and a second centrifugation was performed. This process was repeated twice.

3. Substances Used in Experiment
Calcium chloride (CAS: 10043-52-4, Sigma-Aldrich)
Adenosine-5′-monophosphate disodium salt (CAS: 4578-31-8, Alfa Aesar)
Adenosine 5′-triphosphate disodium salt (51963-61-2, DAEJUNG Reagents)
Adenosine (CAS: 58-61-7, Sigma-Aldrich)
PBS solution (1×pH 7.4, ML 008-01, WELGENE)
Calcium Assay Kit (MAK022, Sigma-Aldrich)

### EXAMPLES

### 1. Example 1

A mixed solution of AMP and calcium chloride was prepared with the concentrations of AMP and calcium chloride varied in 1 ml of ultrapure water. After that, the solution was stood at room temperature for 36 hours to bring about a reaction and form a self-assembled complex.

TABLE 1

| | AMP conc. | ATP conc. |
|---|---|---|
| Example 1-1 | 100 mM | 0 mM |
| Example 1-2 | 99.95 mM | 0.05 mM |
| Example 1-3 | 99.5 mM | 0.5 mM |

### 2. Example 2

A mixed solution of AMP and calcium chloride was prepared with the concentrations of AMP and calcium chloride varied in 5 ml of ultrapure water. After that, the solution was stood at room temperature for 24 hours to bring about a reaction to occur and form a self-assembled complex.

TABLE 2

| | AMP conc. | ATP conc. |
|---|---|---|
| Example 2-1 | 100 mM | 0 mM |
| Example 2-2 | 99 mM | 1 mM |
| Example 2-3 | 95 mM | 5 mM |

### EXPERIMENTAL EXAMPLES

1. Morphological Analysis

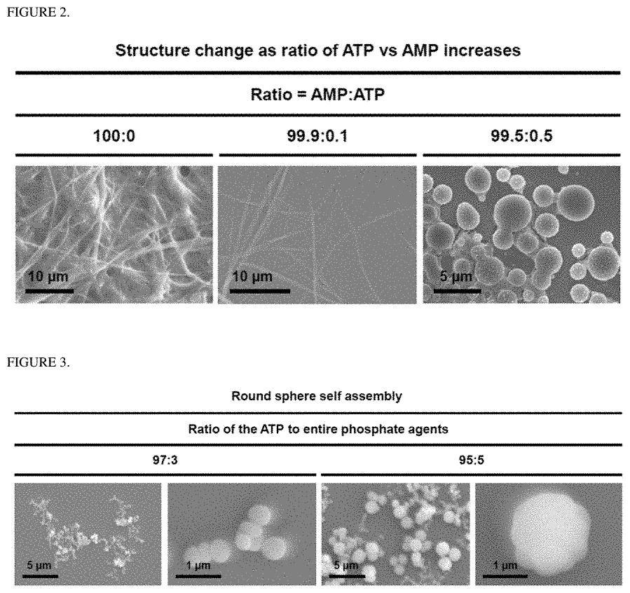

FIGS. 2 and 3 are SEM images of the examples.

Referring to FIGS. 2 and 3, it may be observed that the shape of the self-assembled complex varied by the mixing ratio of the ligand. When the ligand was AMP alone, needle-shaped self-assembled complexes were entangled. However, when ATP was contained at a mixing ratio of 0.1, the self-assembled complex had a longer major axis and a shorter minor axis to form a shape of fibrils. The mixing ratio of ATP being 0.5 caused the self-assembled complex to have a spherical shape. With the mixing ratio of ATP greater than or equal to 0.5, the self-assembled complex no longer had a change in the shape and maintained a spherical shape, or the complex was formed between the spherical shapes.

2. Drug Delivery

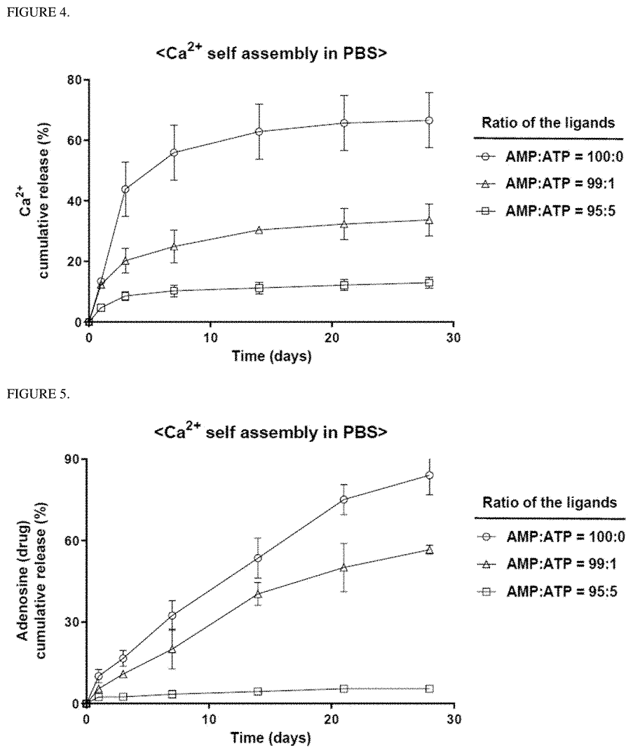

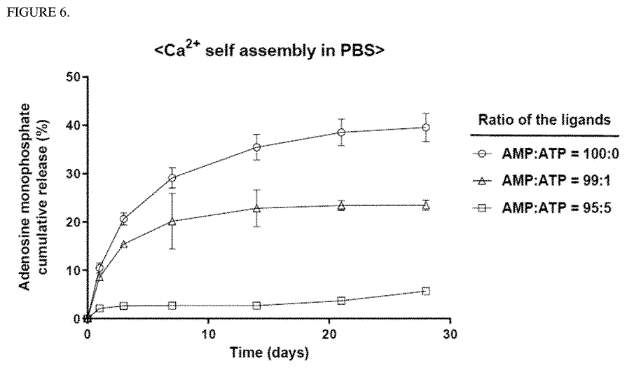

FIGS. 4, 5 and 6 are experimental results for the self-assembly complexes participating in delivery and release of a drug.

With the self-assembled complexes of Example 2 added in a PBS solution, the time of the self-assembled complexes being self-disassembled to release a drug substance was measured. The drug supported in the self-assembled complexes was adenosine.

The self-assembled complexes were put into a PBS solution (5 ml). All of the PBS buffer solution was removed at certain times and the same amount of PBS buffer solution (5 ml) was newly added. This buffer solution was used to establish an environment similar to the in-vivo environment in drug delivery.

After self-disassembly, the amounts of dissolved AMP and adenosine were determined through high performance liquid chromatography (HPLC), and the amount of dissolved calcium was determined using a calcium assay kit.

As can be seen from the figures, the cumulative release amounts of AMP, calcium ion and adenosine constantly increased for about 30 days after the self-disassembly of the self-assembled complexes. In addition, the substances were released at a constant rate until all their contents in the self-assembled complexes became zero.

As a result, it can be seen that the self-assembled complexes have a property of continuously self-disassembling at a certain level.

As for the difference by the mixing ratio of the ligand, when AMP was used alone as the ligand, the release rate decreased with an increase in the mixing ratio of ATP. It is therefore possible to control the rate and duration of substance (drug) delivery by adjusting the mixing ratio of the ligand.

In addition, the results of this experiment imply that the self-assembled complex is capable of delivering a drug or the like in vivo in the same manner as described above.

It should be apparent to those skilled in the present invention that many modifications and variations are possible without departing from the concept or essential features of the present invention. Therefore, the foregoing examples are to be construed as merely illustrative, and not limitative of the present invention. The scope of the present invention is defined by the appended claims rather than the detailed description of the present invention and should be construed as including all changes or modifications derived from the meaning and scope of the claims and their equivalents.

What is claimed is:

1. A self-assembled complex containing a calcium ion, comprising:
a calcium ion; and
at least one ligand,
wherein the ligand and the calcium ion are reversibly self-assembled or self-disassembled,
wherein the self-assembled complex has a shape varying depending on the type of the ligand, or the mixing ratio of the ligand when there are a plurality of ligands.

2. The self-assembled complex containing a calcium ion according to claim 1, wherein the shape of the self-assembled complex changes to a needle, fibrils, and a sphere in sequence with an increase in the concentration of the ligand.

3. The self-assembled complex containing a calcium ion according to claim 2, wherein the needle and the fibrils have a major axis and a minor axis,
wherein the needle has a prolonged shape with the major axis being at least 40- to 60-fold longer than the minor axis,
wherein the fibrils have a shorter minor axis than the needle,
the fibrils having a plurality of branch portions formed on the surface thereof,
wherein the sphere has an average diameter of 0.1 to 10 μm.

4. The self-assembled complex containing a calcium ion according to claim 3, wherein the average diameter of the sphere is less than the length of the major axis of the needle or the fibrils,
wherein the average diameter of the sphere is greater than the length of the minor axis of the needle or the fibrils.

5. The self-assembled complex containing a calcium ion according to claim 1,
wherein the ligand comprises at least any one of phosphate and phosphonate.

6. The self-assembled complex containing a calcium ion according to claim 1, wherein the ligand comprises at least any one selected from the group consisting of AMP, ADP, ATP, TMP, TDP, TTP, CMP, CDP, CTP, GMP, GDP, GTP, UMP, UDP, UTP, DNA, RNA, AEP (2-aminoethylphosphonic acid), TNA (threose nucleic acid), GNA (glycol nucleic acid), HNA (1,5-anhydrohexitol nucleic acid), ANA (1,5-anhydroatritol nucleic acid), FANA (2′-deoxy-2′-fluoroarabino nucleic acid), and CeNa (cyclohexenyl nucleic acid).

7. The self-assembled complex containing a calcium ion according to claim 1, wherein the ligand is AMP,
the self-assembled complex being formed in the shape of a needle.

8. The self-assembled complex containing a calcium ion according to claim 1, wherein the ligand comprises a combination of AMP and ATP,
the AMP and the ATP being included at a molar concentration ratio of 99.99:0.01 to 99.7:0.3,
the self-assembled complex being formed in the shape of fibrils.

9. The self-assembled complex containing a calcium ion according to claim 1, wherein the ligand comprises a combination of AMP and ATP,
the AMP and the ATP being included at a molar concentration ratio of 99.5:0.5 to 96:4,
the self-assembled complex being formed in the shape of a sphere.

10. The self-assembled complex containing a calcium ion according to claim 1, wherein the self-assembled complex is formed by at least any one of π-π interaction and hydrogen bonding between the adjacent ligands; or coordination bonding between the calcium ion and the ligand.

11. The self-assembled complex containing a calcium ion according to claim 1, wherein the calcium ion and the ligand are included at a molar concentration ratio of 5:1 to 1:1.

12. The self-assembled complex containing a calcium ion according to claim 1, wherein the self-disassembly is accelerated under conditions with a buffer solution.

13. The self-assembled complex containing a calcium ion according to claim 1, wherein the self-assembled complex is self-disassembled for 3 to 40 days in vivo or in vitro.

14. The self-assembled complex containing a calcium ion according to claim 1, further comprising an active ingredient,
wherein the active ingredient is supported in the self-assembled complex.

15. The self-assembled complex containing a calcium ion according to claim 14, wherein the active ingredient is at least any one selected from the group consisting of adenosine, guanosine, uridine, cytidine, doxorubicin, a drug, a protein, an organic acid, an organic base, a fragrance, and a dye.

16. The self-assembled complex containing a calcium ion according to claim 14, wherein the active ingredient is released through a self-disassembly of the self-assembled complex,
wherein the active ingredient is released continuously over a release time.

17. The self-assembled complex containing a calcium ion according to claim 15, wherein the release time is 3 to 40 days.

18. The self-assembled complex containing a calcium ion according to claim 1, wherein the self-assembled complex is self-disassembled to release at least any one of the calcium ion and the ligand over a release time,
wherein the release time is adjusted by the mixing ratio of the ligand.

19. The self-assembled complex containing a calcium ion according to claim 1, wherein the self-assembled complex is formed in the shape of a needle,
wherein the self-assembled complex is used as a needle for a microneedle patch.

20. The self-assembled complex containing a calcium ion according to claim 1, wherein the self-assembled complex is used as at least any one selected from a bond regeneration material, an artificial bone material, an in-vivo substance delivery carrier, and an in-vitro substance delivery carrier.
